(19) United States
(12) Patent Application Publication
(10) Pub. No.: US 2011/0256055 A1
(43) Pub. Date: Oct. 20, 2011

(54) APPLICATION OF 99MTC PEPTIDE-BASED COMPOUND AS A BONE MARROW IMAGING AGENT

(75) Inventors: Paul Evans, Amersham (GB); Brian McParland, Amersham (GB); Jose Zubeldia, Las Palmas De Gran Canaria (ES)

(73) Assignee: GE HEALTHCARE LIMITED, LITTLE CHALFONT (GB)

(21) Appl. No.: 13/141,339

(22) PCT Filed: Dec. 15, 2009
(86) PCT No.: PCT/US09/67987

§ 371 Date: Jun. 22, 2011

(30) Foreign Application Priority Data
Dec. 23, 2008 (US) 61140194

(51) Int. Cl. A61K 51/00; G01N 33/566

(52) U.S. Cl. 424/1.11; 435/7.21

(57) ABSTRACT

The present invention relates to methods and materials involved in using peptide-based compounds in bone marrow imaging. More specifically the invention relates to the use of 99mTc peptide-based compounds as targeting vectors that bind to receptors associated with angiogenesis, in particular integrin receptors, e.g. the αvβ3 integrin receptor. Such contrast agents may thus be used for diagnosis of haemolytic anaemia, myeloproliferative disorders, myelofibrosis, selection of biopsy sites, and early detection of skeletal metastatasis as well as detecting avascular necrosis of the femoral heads.

### FIELD OF INVENTION

The present invention relates to methods and materials involved in using peptide-based compounds in bone marrow imaging. More specifically the invention relates to the use of 99mTc peptide-based compounds as targeting vectors that bind to receptors associated with angiogenesis, in particular integrin receptors, e.g. the αvβ3 integrin receptor. Such contrast agents may thus be used for diagnosis of haemolytic anaemia, myeloproliferative disorders, myelofibrosis, selection of biopsy sites, and early detection of skeletal metastatasis as well as detecting avascular necrosis of the femoral heads.

### BACKGROUND OF INVENTION

New blood vessels can be formed by two different mechanisms: vasculogenesis or angiogenesis. Angiogenesis is the formation of new blood vessels by branching from existing vessels. The primary stimulus for this process may be inadequate supply of nutrients and oxygen (hypoxia) to cells in a tissue. The cells may respond by secreting angiogenic factors, of which there are many; one example, which is frequently referred to, is vascular endothelial growth factor (VEGF). These factors initiate the secretion of proteolytic enzymes that break down the proteins of the basement membrane, as well as inhibitors that limit the action of these potentially harmful enzymes. The other prominent effect of angiogenic factors is to cause endothelial cells to migrate and divide. Endothelial cells that are attached to the basement membrane, which forms a continuous sheet around blood vessels on the contralumenal side, do not undergo mitosis. The combined effect of loss of attachment and signals from the receptors for angiogenic factors is to cause the endothelial cells to move, multiply, and rearrange themselves, and finally to synthesise a basement membrane around the new vessels.

Angiogenesis is prominent in the growth and remodelling of tissues, including wound healing and inflammatory processes. Tumors must initiate angiogenesis when they reach millimetre size in order to keep up their rate of growth. Angiogenesis is accompanied by characteristic changes in endothelial cells and their environment. The surface of these cells is remodelled in preparation for migration, and cryptic structures are exposed where the basement membrane is degraded, in addition to the variety of proteins which are involved in effecting and controlling proteolysis. In the case of tumours, the resulting network of blood vessels is usually disorganised, with the formation of sharp kinks and also arteriovenous shunts. Inhibition of angiogenesis is also considered to be a promising strategy for antitumour therapy. The transformations accompanying angiogenesis are also very promising for diagnosis, an obvious example being malignant disease, but the concept also shows great promise in inflammation and a variety of inflammation-related diseases, including atherosclerosis, the macrophages of early atherosclerotic lesions being potential sources of angiogenic factors. These factors are also involved in re-vascularisation of infarcted parts of the myocardium, which occurs if a stenosis is released within a short time.

Further examples of undesired conditions that are associated with neovascularization or angiogenesis, the development or proliferation of new blood vessels are shown below. Reference is also made in this regard to WO 98/47541.

Diseases and indications associated with angiogenesis are e.g. different forms of cancer and metastasis, e.g. breast, skin, colorectal, pancreatic, prostate, lung or ovarian cancer.

Other diseases and indications are inflammation (e.g. chronic), atherosclerosis, rheumatoid arthritis and gingivitis.

Further diseases and indications associated with angiogenesis are arteriovenous alformations, astrocytomas, choriocarcinomas, glioblastomas, gliomas, hemangiomas (childhood, capillary), hepatomas, hyperplastic endometrium, ischemic myocardium, endometriosis, Kaposi sarcoma, macular degeneration, melanoma, neuroblastomas, occluding peripheral artery disease, osteoarthritis, psoriasis, retinopathy (diabetic, proliferative), scleroderma, seminomas and ulcerative colitis.

Angiogenesis involves receptors that are unique to endothelial cells and surrounding tissues. These markers include growth factor receptors such as VEGF and the Integrin family of receptors. Immunohistochemical studies have demonstrated that a variety of integrins perhaps most importantly the αᵥ class are expressed on the apical surface of blood vessels [Conforti, G., et al. (1992) Blood 80: 37-446] and are available for targeting by circulating ligands [Pasqualini, R., et al. (1997) Nature Biotechnology 15: 542-546]. The α5β1 is also an important integrin in promoting the assembly of fibronectin matrix and initiating cell attachment to fibronectin. It also plays a crucial role in cell migration [Bauer, J. S., (1992) J. Cell Biol. 116: 477-487] as well as tumour invasion and metastasis [Gehlsen, K. R., (1988) J. Cell Biol. 106: 925-930].

The integrin αvβ3 is one of the receptors that is known to be associated with angiogenesis. Stimulated endothelial cells appear to rely on this receptor for survival during a critical period of the angiogeneic process, as antagonists of the αvβ3 integrin receptor/ligand interaction induce apoptosis and inhibit blood vessel growth.

Integrins are heterodimeric molecules in which the α- and β-subunits penetrate the cell-membrane lipid bilayer. The α-subunit has four Ca²⁺ binding domains on its extracellular chain, and the β-subunit has a number of extracellular cysteine-rich domains.

Many ligands (eg. fibronectin) involved in cell adhesion contain the tripeptide sequence arginine-glycine-aspartic acid (RGD). The RGD sequence appears to act as a primary recognition site between the ligands presenting this sequence and receptors on the surface of cells. It is generally believed that secondary interactions between the ligand and receptor enhance the specificity of the interaction. These secondary interactions might take place between moieties of the ligand and receptor that are immediately adjacent to the RGD sequence or at sites that are distant from the RGD sequence.

RGD peptides are known to bind to a range of integrin receptors and have the potential to regulate a number of cellular events of significant application in the clinical setting. (Ruoslahti, J. Clin. Invest., 87: 1-5 (1991)). Perhaps the most widely studied effect of RGD peptides and mimetics thereof relate to their use as anti-thrombotic agents where they target the platelet integrin GpIIbIIIa.

Inhibition of angiogenesis in tissues by administration of either an αvβ3 or αvβ5 antagonist has been described in for example WO 97/06791 and WO 95/25543 using either antibodies or RGD containing peptides. EP 578083 describes a series of mono-cyclic RGD containing peptides and WO 90/14103 claims RGD-antibodies. Haubner et al. in the J. Nucl. Med. (1999); 40: 1061-1071 describe a new class of tracers for tumour targeting based on monocyclic RGD containing peptides. Biodistribution studies using whole-body autoradiographic imaging revealed however that the ¹²⁵I-labelled peptides had very fast blood clearance rates and predominantly hepatobiliary excretion routes resulting in high background.

Cyclic RGD peptides containing multiple bridges have also been described in WO 98/54347 and WO 95/14714. Peptides derived from in vivo biopanning (WO 97/10507) have been used for a variety of targeting applications. The sequence CDCRGDCFC (RGD-4C), has been used to target drugs such as doxirubicin (WO 98/10795), nucleic acids and adenoviruses to cells (see WO 99/40214, WO 99/39734, WO 98/54347, WO 98/54346, U.S. Pat. No. 5,846,782). Peptides containing multiple cysteine residues do however suffer from the disadvantage that multiple disulphide isomers can occur. A peptide with 4 cysteine residues such as RGD-4C has the possibility of forming 3 different disulphide folded forms. The isomers will have varying affinity for the integrin receptor as the RGD pharmacophore is forced into 3 different conformations.

Further examples of RGD comprising peptide-based compounds are found in PCT/N001/00146 and PCT/N001/00390.

There is a need however for evaluating bone marrow that overcomes marrow sampling errors by giving a total body view of functioning marrow. Bone marrow aspiration and biopsy are known techniques for evaluating bone marrow, but this evaluation is limited to a small part of the total blood-forming organ. Radionuclide bone marrow imaging is a simply technique that overcomes marrow sampling errors by giving a total body view of functioning marrow.

By utilizing 99mTc peptide-based compounds as targeting vectors that bind to receptors associated with angiogenesis, in particular integrin receptors, e.g. the αvβ3 integrin receptor, the present invention assesses the need for evaluating bone marrow.

### SUMMARY OF THE INVENTION

This document provides methods and materials involved in using peptide-based compounds in bone marrow imaging. As described herein, the expression of αvβ3 integrin polypeptides on bone marrow can aid in overcoming marrow sampling errors by giving a full and complete body view of functioning marrow.

Bone marrow aspiration and biopsy are excellent techniques for evaluating bone marrow, but this evaluation is limited to a small part of the total blood-forming organ. Radionuclide bone marrow imaging is a simple technique that overcomes marrow sampling errors by giving a total body view of functioning marrow. Furthermore, the procedure is noninvasive and provides an atraumatic method for evaluating a number of clinical problems including a discrepancy between bone marrow histology and clinical status (possible marrow sampling error), the determination of the amount of active marrow after radiation and chemotherapy when further therapy is being considered, detection of sites of extramedullary hematopoiesis, location of the optimal sites for bone marrow biopsy, the diagnosis and staging of diffuse hematologic disorders, detection of metastases, the diagnosis of bone marrow infarcts in hemolytic anemias, and detecting avascular necrosis of the femoral heads.

There are two major classes of bone marrow agents, those that are incorporated into the erythroid precursors such as radioiron, and colloids that are taken up by the reticuloendothelial system (RES). The present invention is directed to erythroid precursors such as radioiron since the 99mTc peptide-based compound is not based on albumin thus giving this product a wider application compared to 99mTc-albumin nanocolloid. Furthermore, the in vivo characteristics of 99mTc-NC100692 provide imaging properties that are superior to present bone marrow imaging radiopharmaceuticals.

Unless otherwise defined, all technical and scientific terms used herein have the same meaning as commonly understood by one skilled in the art to which this invention pertains.

### DETAILED DESCRIPTION OF THE INVENTION

Radionuclide imaging of bone marrow is useful to visualise defects in bone marrow distribution, non-uniform marrow distribution or expansion of active marrow into long bones, occurring in haemolytic anaemia, myeloproliferative disorders or myelofibrosis. Bone marrow imaging also aids in the selection of biopsy sites as well as in the early detection of skeletal metastasis.

This document provides methods and materials involved in using peptide-based compounds in bone marrow imaging. As described herein, the expression of αvβ3 integrin polypeptides on bone marrow can aid in overcoming marrow sampling errors by giving a full and complete body view of functioning marrow.

The invention relates to the use of 99mTc peptide-based compounds as targeting vectors that bind to receptors associated with angiogenesis, in particular integrin receptors, e.g. the αvβ3 integrin receptor. Such contrast agents may thus be used for diagnosis of haemolytic anaemia, myeloproliferative disorders, myelofibrosis, selection of biopsy sites, and early detection of skeletal metastatasis as well as detecting avascular necrosis of the femoral heads.

Any method can be used to determine whether or not αvβ3 integrin polypeptides are expressed on bone marrow. For example, bone marrow tissue can be contacted with a molecule that binds to cells expressing αvβ3 integrin polypeptides. The level of binding of such a molecule to the bone marrow tissue can be detected. The presence of the molecule bound to the tissue can indicate that the tissue has αvβ3 integrin polypeptides. The absence of the molecule bound to the tissue or the presence of a barely detectable level of the molecule bound to the tissue can indicate that the tissue expresses no, or low level of, αvβ3 integrin polypeptides.

An αvβ3 integrin polypeptide can be any integrin polypeptide having αv and β3 polypeptide subunits. Examples of integrin αv polypeptides include human integrin αv polypeptides (e.g. human integrin αv polypeptides set forth under GenBank® GI nos. gi4504763 or gi466372). Examples of integrin β3 polypeptides include human integrin αv polypeptides (e.g. human integrin β3 polypeptides set forth under GenBank® GI nos. gi54124349 or gi386833). Examples of integrin αv and β3 Polypeptides also include variants of integrin αv and β3 polypeptides as well as homologs and orthologs of integrin αv and β3 polypeptides.

Any molecule that binds to cells having αvβ3 integrin polypeptides and that can be detected when bound to αvβ3 integrin polypeptides on cells can be used to assess the presence, absence, or low level of αvβ3 integrin polypeptides on bone marrow. For example, RGD peptides, small molecule αvβ3 integrin anatagonists, lectins, or anti-αvβ3 integrin antibodies can be used to determine whether or not αvβ3 integrin polypeptides are expressed by bone marrow.

An RGD peptide can be any polypeptide comprising an arginine, glycine, aspartic acid amino acid sequence. Examples of an RGD peptide include, without limitation 99mTc-NC100692 (Mousa, J. Cardiovasc. Pharmacol. 45:462 (2005). An RGD peptide can be a monomeric polypeptide or a multimeric (e.g. dimeric) polypeptide. An RGD peptide also can be cyclic and can be stabilized (e.g. by disulphide bonds). Further, an RGD peptide can be modified to be pegylated or covalently attached to oligomers, such as short, amphiphilic oligomers that enable oral administration or improve the pharmacokinetic or pharmacodynamic profile of a conjugated RGD peptide. The oligomers can comprise water soluable polyethylene glycol (PEG) and lipid soluable alkyls.

An antibody can be, without limitation, a polyclonclonal, monoclonal, human, humanized chimeric, or single-chain antibody, or an antibody fragment having binding activity. An antibody be of any type (IgG, IgM, IgD, or IgY), class (IgG4 or IgA2), or subclass. In addition an antibody can be human, rabbit, sheep, or goat. An antibody can be naturally occurring, recombinant, or synthetic. Antibodies can be be generated and purified using any suitable methods known in the art.

A molecule that binds to cells having αvβ3 integrin polypeptides can be labelled for detection. A molecule capable of binding to cells having αvβ3 integrin polypeptides can also be detected indirectly using a labelled molecule that binds to cells having αvβ3 integrin polypeptides.

Any method can be used to determine whether or not a molecule binds to cells expressing αvβ3 integrin polypeptides. Additionally, a method can be used to determine the level of expression of an αvβ3 integrin polypeptide presenting bone marrow cells or tissues.

This document provides methods and materials to assist medical or research professionals in determining whether bone marrow tissue is defective or not. Medical professionals can be, but not limited to, doctors, nurses, medical lab technologists, and pharmacists. Research professionals can be, but not limited to, investigators, research technicians, postdoctoral trainees, and graduate students.

Any appropriate method can be used to communicate information to another person (professional).

When the αvβ3 integrin polypeptide is found in bone marrow of a subject then this is an indication of abnormal. Or when the amount of αvβ3 is more than a trace amount in the bone marrow of a subject then this is abnormal. If the αvβ3 integrin polypeptide expression is of no significant value in the bone marrow tissue or cell than this is deemed normal.

One embodiment of the present invention depicts a method for assessing abnormalities in bone marrow tissue, said method comprising determining whether or not said bone marrow tissue or cell expresses an αvβ3 integrin polypeptide, wherein the expression of said αvβ3 integrin polypeptide indicates that said bone marrow is abnormal, and wherein the expression of little or no αvβ3 integrin polypeptide indicates that said bone marrow is normal.

Another method of the invention presents bone marrow tissue being human bone marrow tissue. The term tissue encompasses cells of a mammal or human for purposes herein.

The present invention further discloses a method wherein said determining step comprises contacting tissue of bone marrow with a labelled molecule having the ability to bind to said αvβ3 integrin polypeptide.

Yet another embodiment of the invention includes a method wherein said labelled molecule is an antibody.

Still another embodiment includes a method wherein said labelled molecule is an RGD peptide and further wherein said labelled molecule is 99mTc-NC-100692.

Another embodiment includes a method wherein said labelled molecule is administered to a mammal having bone marrow tissue.

Still another embodiment of the present invention presents a method for assessing bone marrow tissue, said method comprising determining whether or not said bone marrow tissue lacks expression of an αvβ3 integrin polypeptide, wherein the lack of expression of said αvβ3 integrin polypeptide indicates that said bone marrow tissue is normal.

Another embodiment includes a method wherein said bone marrow tissue is a human bone marrow tissue.

Still another embodiment of the invention shows a method wherein said determining step comprises contacting tissue of said bone marrow tissue with a labelled molecule having the ability to bind to said αvβ3 integrin polypeptide.

A further embodiment of the present invention discloses a labelled molecule as an antibody and further wherein said labelled molecule is an RGD peptide and further wherein the RGD peptide is 99mTc-NC-100692.

Another embodiment of the invention is a method wherein said labelled molecule is administered to a mammal having bone marrow tissue.

The present invention will now be further illustrated by way of the following non-limiting examples.

### EXAMPLES

### Example 1

### Detecting Defective Bone Marrow Tissue in a Clinical Study

The following clinical study is performed to verify the improved sensitivity and specificity of using αvβ3 integrin expression to differentiate between normal and defective bone marrow. The primary endpoint is diagnostic accuracy. Patients are recruited from an active patient population. Management algorithms are typically based on clinical experience, radiologic appearance, rate of change in appearance, and patient preference. There are a number of other tests used to help guide management including CT contrast enhancement, bone marrow aspiration, biopsy and radionuclide bone marrow imaging.

Subjects are patients presenting for evaluation of abnormal bone marrow. After consent, they are randomized to either of two study groups: one, with no intervention (physician usual care); and the second, using clinical practice but including testing αvβ3 integrin expression. The diagnostic accuracy of the αvβ3 integrin expression testing is determined by following these groups until diagnostic of the bone marrow is made. Image analysis is performed by one of three experienced radiologists. They are blinded to the subject's clinical history and other test results. Diagnostic accuracy is assessed in comparison to the final diagnostic of the bone marrow treated by usual care. Sensitivity, specificity, positive, and negative predictive values are calculated.

### Specific Embodiments, Citation of References

The present invention is not to be limited in scope by specific embodiments described herein. Indeed, various modifications of the inventions in addition to those described herein will become apparent to those skilled in the art from the foregoing description and accompanying figures. Such modifications are intended to fall within the scope of the appended claims.

Various publications and patent applications are cited herein, the disclosures of which are incorporated by reference in their entireties.

1. A method for assessing abnormalities in bone marrow tissue, said method comprising:
(i) contacting tissue of said bone marrow with a radiolabelled molecule having the ability to bind to cells which express an αvβ3 integrin polypeptide;
(ii) detecting the level of binding of said radiolabelled molecule to said bone marrow tissue, wherein the binding of said radiolabelled molecule to said tissue indicates that the tissue has cells expressing αvβ3 integrin polypeptides, and the absence of detectable binding indicates that the tissue lacks such cells;
(iii) determining that, where there is detectable binding, that said bone marrow is abnormal, and where there is no detectable binding that said bone marrow is normal.

2. The method of claim 1, wherein said bone marrow tissue is a human bone marrow tissue.

3. (canceled)

4. The method of claim 1, wherein said radiolabelled molecule is an antibody.

5. The method of claim 1, wherein said radiolabelled molecule is an RGD peptide.

6. The method of claim 1, wherein said radiolabelled molecule is ^{99m}Tc-NC-100692.

7. The method of claim 1, wherein said radiolabelled molecule is administered to a mammal having bone marrow tissue.

8-14. (canceled)
